SEQUENCE LISTING (5 sequences)

SEQ ID NO: 1 - DNA, 307 bp, Unknown
  a bacterium belonging to Atopobiaceae
gatgaacgctggcggcgcgcctaacacatgcaagtcgaacgagaagcacctcttcggaggtgtggatagtggcgaacggctgagtaacacgtgggcaacctgcccctctcatcgggatagcctcgggaaaccggggataataccggatactccaacgctttcgcatggaagcgttgggaaagctctggcggagagggatgggcccgcggcctgttagctagttggcggggtaacggcccaccaaggcttctatgggtagccgggttgagagaccgaccggccagattgggactgagacacggcccag

SEQ ID NO: 2 - DNA, 20 bp, Artificial Sequence
  Universal primer 27Fmod
agrgtttgatymtggctcag

SEQ ID NO: 3 - DNA, 19 bp, Artificial Sequence
  Universal primer 338R
tgctgcctcccgtaggagt

SEQ ID NO: 4 - DNA, 69 bp, Artificial Sequence
  Primer for adding adaptor and index sequences to be required for sequencing
  misc_feature (30)..(37): n is a, c, g, or t
aatgatacggcgaccaccgagatctacacnnnnnnnntatggtaattgtagrgtttgatymtggctcag

SEQ ID NO: 5 - DNA, 63 bp, Artificial Sequence
  Primer for adding adaptor and index sequences to be required for sequencing
  misc_feature (25)..(32): n is a, c, g, or t
caagcagaagacggcatacgagatnnnnnnnnagtcagtcagcctgctgcctcccgtaggagt

(19) United States
(12) Patent Application Publication
(10) Pub. No.: US 2021/0128645 A1
(43) Pub. Date: May 6, 2021

(54) INTESTINAL MICROBE HAVING D-PSICOSE-RESPONSIVE PROLIFERATION

(71) Applicant: Keio University, Tokyo (JP)

(72) Inventors: Shinji FUKUDA, Tsuruoka (JP); Nao TAKEUCHI, Tsuruoka (JP)

(21) Appl. No.: 17/144,868

(22) Filed: Jan. 8, 2021

Related U.S. Application Data
(63) Continuation of application No. PCT/JP2019/027371, filed on Jul. 10, 2019.

(30) Foreign Application Priority Data
Jul. 10, 2018 (JP) 2018-131052

(51) Int. Cl. A61K 35/741; C12N 1/20; A61P 3/04

(52) U.S. Cl. CPC A61K 35/741; A61K 2035/115; A61P 3/04; C12N 1/20

(57) ABSTRACT

The present invention provides a novel drug or food that can produce desired effects. More specifically, the present invention provides: an intestinal microbe having D-psicose-responsive proliferation; a drug or food containing the intestinal microbe having D-psicose-responsive proliferation; a biological function-improving agent containing the intestinal microbe having D-psicose-responsive proliferation; an intestinal microbe culturing method that includes culturing the intestinal microbe in a D-psicose-containing culture medium; a screening method for a biological function-improving agent using the intestinal microbe having D-psicose-responsive proliferation; and the like.

### TECHNICAL FIELD

Many beverages contain artificial sweeteners, and consumers expect health benefits of artificial sweeteners from phrases like zero calories. However, artificial sweeteners have concerns of their safety and effects since it has been suggested that artificial sweeteners may change the composition and function of intestinal microbiota to cause impaired glucose tolerance (Non Patent Literature 1). There is thus a need of functional sweeteners that replace artificial sweeteners.

It has been reported that rare sugar D-psicose (also named D-allulose) has almost zero calories while maintaining sweetness equivalent to about 70% the sweetness of sucrose, and yet has biological function-improving effects, such as improvement of glucose tolerance and an anti-obesity effect (Non Patent Literature 2 and Non Patent Literature 3). It has also been reported that a portion of D-psicose passes through the gastrointestinal tract to the large intestine without being absorbed (Non Patent Literature 4 and Non Patent Literature 5).

### CITATION LIST

### Non Patent Literature

Non Patent Literature 1: Suez J, Korem T, Zeevi D, Zilberman-Schapira G, Thaiss C A, Maza O, et al. Artificial sweeteners induce glucose intolerance by altering the gut microbiota. Nature. 2014;514(7521):181-6.
Non Patent Literature 2: Iwasaki Y, Sendo M, Dezaki K, Hira T, Sato T, Nakata M, et al. GLP-1 release and vagal afferent activation mediate the beneficial metabolic and chronotherapeutic effects of D-allulose. Nat Commun. 2018;9(1):113.
Non Patent Literature 3: Hossain A, Yamaguchi F, Hirose K, Matsunaga T, Sui L, Hirata Y, et al. Rare sugar D-psicose prevents progression and development of diabetes in T2DM model Otsuka Long-Evans Tokushima Fatty rats. Drug Des Devel Ther. 2015;9:525-35.
Non Patent Literature 4: Matsuo T, Tanaka T, Hashiguchi M, Izumori K, Suzuki H. Metabolic effects of D-psicose in rats:Studies on faecal and urinary excretion and caecal fermentation. Asia Pac J Clin Nutr. 2003;12(2):225-31. Non Patent Literature 5: Iida T, Hayashi N, Yamada T, Yoshikawa Y, Miyazato S, Kishimoto Y, et al. Failure of d-psicose absorbed in the small intestine to metabolize into energy and its low large intestinal fermentability in humans. Metabolism. 2010;59(2):206-14.

### SUMMARY OF INVENTION

The Sequence Listing created on Jan. 8, 2021 with a file size of 3 KB, and filed herewith in ASCII text file format as the file entitled “42V0586.TXT,” is hereby incorporated by reference in its entirety.

### Technical Problem

An objective of the present invention is to provide a novel drug or food that can produce desired effects.

### Solution to Problem

The inventors of the present invention have carried out intensive studies and, as a result, have found out that a particular microbe present in the intestine may be a key player in the biological function-improving effects of D-psicose.

That is, the present invention is as follows.
[1] An intestinal microbe having D-psicose-responsive proliferation.
[2] The intestinal microbe according to [1], wherein the intestinal microbe belongs to the family Atopobiaceae.
[3] The intestinal microbe according to [1] or [2], wherein the intestinal microbe has a 16S rRNA gene containing a base sequence having 90% or more identity to a base sequence of SEQ ID NO:1.
[4] The intestinal microbe according to any one of [1] to
[3], wherein the intestinal microbe is an intestinal microbe obtained from feces of a mammal to which D-psicose has been administered.
[5] The intestinal microbe according to [2], wherein the intestinal microbe belonging to the family Atopobiaceae is Atopobium parvulum.
[6] A drug or food comprising an intestinal microbe having D-psicose-responsive proliferation.
[7] A biological function-improving agent comprising an intestinal microbe having D-psicose-responsive proliferation.
[8] The agent according to [7], wherein the biological function-improving agent is a prophylactic or therapeutic agent for metabolic disorder.
[9] The agent according to [7] or [8], wherein the biological function-improving agent is an anti-obesity agent.
[10] The agent according to [7] or [8], wherein the biological function-improving agent is an anti-diabetic agent.
[11] The agent according to any one of [7] to [10], wherein the biological function-improving agent is a composition for oral or rectal administration.
[12] An intestinal microbe culturing method comprising culturing an intestinal microbe in a D-psicose-containing culture medium.
[13] A culture medium comprising D-psicose.
[14] An intestinal microbial culture comprising D-psicose and an intestinal microbe.
[15] A screening method for a substance having biological function-improving effects, the method comprising:

(1) brining investigational substances into contact with an intestinal microbe having D-psicose-responsive proliferation;

(2) evaluating the number of the intestinal microbes having D-psicose-responsive proliferation; and

(3) selecting, as the substance having biological function-improving effects, an investigational substance that increases the number of the intestinal microbes having D-psicose-responsive proliferation.
[16] An intestinal microbe growth enhancing agent comprising D-psicose, wherein the intestinal microbe belongs to the family Atopobiaceae.
[17] The agent according to claim [16], wherein the intestinal microbe belonging to the family Atopobiaceae is Atopobium parvulum.
[18] The agent according to claim [16], wherein the intestinal microbe growth enhancing agent is a composition for oral or rectal administration.
[19] The agent according to claim [17], wherein the intestinal microbe growth enhancing agent is a composition for oral or rectal administration.
[20] A biological function-improving agent comprising D-psicose and an intestinal microbe belonging to the family Atopobiaceae.
[21] The agent according to claim [20], wherein the biological function-improving agent is a prophylactic or therapeutic agent for metabolic disorder.
[22] The agent according to claim [20], wherein the biological function-improving agent is an anti-obesity agent.
[23] The agent according to claim [20], wherein the biological function-improving agent is an anti-diabetic agent.
[24] The agent according to claim [20], wherein the biological function-improving agent is a composition for oral or rectal administration.
[25] The agent according to claim [21], wherein the biological function-improving agent is a composition for oral or rectal administration.
[26] The agent according to claim [22], wherein the biological function-improving agent is a composition for oral or rectal administration.
[27] The agent according to claim [23], wherein the biological function-improving agent is a composition for oral or rectal administration.

### Advantageous Effects of Invention

The intestinal microbe according to the present invention is useful as, for example, a drug or food, or a biological function-improving agent (e.g., an anti-obesity agent or an anti-diabetic agent).

The intestinal microbe growth enhancing agent according to the present invention is useful as, for example, a drug or food, or a biological function-improving agent (e.g., an anti-obesity agent or an anti-diabetic agent).

The culturing method according to the present invention is useful for, for example, proliferation and maintenance of intestinal microbes having D-psicose-responsive proliferation.

The screening method according to the present invention is useful for, for example, development of a novel drug or food having biological function-improving effects like D-psicose, and development of a drug or food having multiple effects, specifically, biological function-improving effects like D-psicose in combination with existing effects.

### BRIEF DESCRIPTION OF DRAWINGS

FIG. 1 shows the schedule of an animal experiment. Eight-week-old (week 0) male C57BL/6J mice (total 4 groups, n=7) were fed a high-fat diet and tap water (control) or D-psicose-containing water, or antibiotics in addition to these (Control+Abx, D-psicose+Abx). During the experimental period, the body weight of the mice, the water intake, and the food intake were measured weekly, and the feces were collected. The samples at weeks 0 and 5 were used for analysis. The fasting blood glucose level was measured at week 9. The mice were dissected after 11 weeks.

FIG. 2 shows the suppression of body weight gain due to the intake of D-psicose and the reduction in the suppressive effect due to antibiotic treatment. The average (g) of the body weight gain at week 5 (13 weeks old) from start of rearing (8 weeks old) is shown (n =7). The error bars represent standard deviations (SDs). Control (white): high-fat diet+tap water, D-psicose (black): high-fat diet +5.0% D-psicose, Abx−: without antibiotic treatment, Abx+: with antibiotic treatment. The antibiotics were 0.1% ampicillin sodium, 0.1% neomycin sulfate, and 0.05% vancomycin hydrochloride. The Student's t-test was used in significance testing. A p-value of 0.05 or less is considered significant and indicated by*in the figure.

FIG. 3 shows the suppression of an increase in fasting blood glucose level due to the intake of D-psicose and the reduction in the suppressive effect due to antibiotic treatment. The 15-hour fasting blood glucose level (mg/dl) at week 9 after start of rearing is shown (n=7). The error bars represent standard deviations (SDs). Control (white): high-fat diet+tap water, D-psicose (black): high-fat diet +5.0% D-psicose, Abx−: without antibiotic treatment, Abx+: with antibiotic treatment. The antibiotics were 0.1% ampicillin sodium, 0.1% neomycin sulfate, and 0.05% vancomycin hydrochloride. The Student's t-test was used in significance testing. A p-value of 0.05 or less is considered significant and indicated by*in the figure.

FIG. 4 shows the growth of the genus Atopobium with the intake of D-psicose. The relative abundance of the genus Atopobium in the fecal samples at week 5 from start of rearing is shown. The relative abundance is calculated as the percentage of the genus Atopobium in the total reads. Control (white): high-fat diet+tap water, D-psicose (black): high-fat diet +5.0% D-psicose. The Mann-Whitney U test was used in significance testing. A p-value of 0.001 or less is considered significant and indicated by P<0.001: in the figure.

FIG. 5 shows the base sequence (SEQ ID NO:1) of a 16S rRNA gene of the operational taxonomic unit (OTU) of the intestinal microbe found in the present invention. In two times of animal tests (total n=12), OTUs having the same base sequence as that base sequence were detected.

FIG. 6 shows the suppression of body weight gain due to Atopobium parvulum colonization. The average body weight (g) for 9 weeks from start of rearing (8 weeks old) is shown (n=5). The error bars represent standard deviations (SDs). GF (white circle): germ-free mice, B. thetaiotaomicron (black triangle): mice colonized with Bacteroidetes thetaiotaomicron (JCM 5827), A. parvulum (black circle): mice colonized with Atopobium parvulum (JCM 10300). When a significant difference with P<0.05 is observed by two-way analysis of variance (two-way ANOVA), multiple comparison is performed by the Tukey's test. P<0.001 is indicated by***in the figure.

FIG. 7 shows the suppression of the epididymal adipose tissue weight due to Atopobium parvulum colonization. The average weight (g) of the epididymal adipose tissue for 9 weeks from start of rearing (8 weeks old) is shown (n=5). The error bars represent standard deviations (SDs). GF (white): germ-free mice, B. thetaiotaomicron (gray): mice colonized with Bacteroidetes thetaiotaomicron (JCM 5827), A. parvulum (black): mice colonized with Atopobium parvulum (JCM 10300). When a significant difference with P<0.05 is observed by two-way analysis of variance (two-way ANOVA), multiple comparison is performed by the Tukey's test. P<0.01 is indicated by**in the figure.

### DESCRIPTION OF EMBODIMENTS

1. Intestinal Microbe having D-Psicose-Responsive Proliferation

The present invention provides an intestinal microbe having D-psicose-responsive proliferation.

Intestinal microbes having D-psicose-responsive proliferation are intestinal microbes that significantly grow in the intestine of mammals in association with the intake of D-psicose in the mammals.

An intestinal microbe according to the present invention may be an intestinal microbe belonging to the family Atopobiaceae. Examples of known intestinal microbes belonging to the family Atopobiaceae include microbes belonging to the genus Atopobium (e.g., Atopobium parvulum) and microbes belonging to the genus Olsenella (e.g., Olsenella umbonata).

The intestinal microbe according to the present invention can be characterized by having a 16S rRNA gene containing a base sequence having 90% or more identity to the base sequence of SEQ ID NO:1. Preferably, the identity of the base sequence may be 91% or more, 92% or more, 93% or more, 94% or more, 95% or more, 96% or more, 97% or more, 98% or more, 99% or more, or 100%, but preferably 97% or more, 98% or more, 99% or more, or 100%. It is said that OTUs (e.g., the base sequence of the 16S rRNA gene) having 97% or more identity are assigned to evolutionary the same microbial species. It can therefore be considered that intestinal microbes having a 16S rRNA gene containing a base sequence having 97% or more identity to the base sequence of SEQ ID NO:1 belong to the same microbial species.

The identity % of polynucleotides (genes) can be calculated by algorithm BLAST. More specifically, the identity % of polynucleotides can be calculated by using default settings (scoring parameters: match/mismatch scores=1, -2; gap costs =linear) in algorithm BLAST available from NCBI.

The intestinal microbe according to the present invention can further exhibit biological function-improving effects. The biological function-improving effects exhibited by the intestinal microbe according to the present invention are the same as the biological function-improving effects of D-psicose. Examples of the biological function-improving effects of D-psicose include the prophylactic or therapeutic effect on metabolic disorder (e.g., the anti-obesity effect, the glucose tolerance improving effect) and the anti-atherosclerotic effect. Therefore, the intestinal microbe according to the present invention can be characterized by these effects.

The intestinal microbe according to the present invention can be obtained from, for example, feces of a mammal. Examples of the mammal include primates (e.g., human, monkey), rodents (e.g., mouse, rat, guinea pig, rabbit), dog, cat, cattle, horse, and pig. The mammal is preferably a primate or rodent, more preferably human or mouse. From the viewpoint of ease of clinical application, the mammal is still more preferably human. It can be said that the intestinal microbe according to the present invention is a microbe present in the intestine of these mammals. The methods for collecting intestinal microbes from feces of mammals are well known (e.g., (i) Fukuda S et al., J Vet Med Sci.,2002 Nov; 64(11): 987-92, (ii) Fukuda S et al., J Gen Appl Microbiol., 2005 April; 51(2): 105-13, (iii) Sasaki D et al., Sci Rep., 2018 Jan. 11; 8(1): 435).

To efficiently obtain the intestinal microbe according to the present invention, the intestinal microbe according to the present invention can be obtained from feces of a mammal that has received D-psicose (or to which D-psicose has been administered). The amount of intake of D-psicose is not limited as long as it is an amount sufficient to cause proliferation of the intestinal microbe according to the present invention (in other words, it is an amount sufficient to exhibit the effects of D-psicose). The amount of intake of D-psicose may vary depending on factors, such as target mammalian species, intake frequency, and intake period, but the amount of intake per day is, for example, 0.05 to 100 g/kg (body weight), preferably 0.1 to 50 g/kg (body weight), more preferably 0.2 to 50 g/kg (body weight).

2. Drug or Food and Biological Function-Improving Agent Containing Intestinal Microbe having D-Psicose-Responsive Proliferation

The present invention provides a drug or food and a biological function-improving agent (hereinafter referred to as a product according to the present invention as necessary). The product according to the present invention contains an intestinal microbe having D-psicose-responsive proliferation.

The number of intestinal microbes having D-psicose-responsive proliferation contained in the product according to the present invention is not limited as long as the intestinal microbe having D-psicose-responsive proliferation has beneficial effects on mammals that have received the intestinal microbe. Since the intestinal microbe having D-psicose-responsive proliferation is a microbe originally present in the intestine, a small amount of intake of the intestinal microbe in mammals still allows colonization and proliferation in the intestine to exhibit the effects, depending on factors, such as intestinal environment and individual differences. Therefore, the intestinal microbe having D-psicose-responsive proliferation contained in the product according to the present invention can exhibit their effects even at a small amount. However, the number of intestinal microbes having D-psicose-responsive proliferation contained in the product according to the present invention is preferably larger than or equal to a certain level in order to, for example, improve the possibility of colonization in the intestine and readily exhibit the effects. For the intestinal microbe having D-psicose-responsive proliferation, the number of intake of the intestinal microbe per time is, for example, 1×10⁵ to 1×10⁹ cells/kg (body weight), preferably 5×10⁵ to 5×10⁸ cells/kg (body weight), more preferably 1×10⁶ to 1×10⁸ cells/kg (body weight). In the product according to the present invention, the number of intestinal microbes in such a range can be contained in one or two or more (e.g., 2 to 6) solid preparations (e.g., capsules, tablets).

The product according to the present invention may be provided in the form of composition. A composition according to the present invention containing the intestinal microbe having D-psicose-responsive proliferation may further contain D-psicose.

For example, the product according to the present invention, when being provided in the form of pharmaceutical composition, may contain a pharmaceutically acceptable carrier in addition to the intestinal microbe having D-psicose-responsive proliferation. Examples of the pharmaceutically acceptable carrier include, but are not limited to, vehicles, such as sucrose, starch, mannit, sorbit, lactose, glucose, cellulose, talc, calcium phosphate, and calcium carbonate; binders, such as cellulose, methyl cellulose, hydroxypropyl cellulose, polypropyl pyrrolidone, gelatin, gum arabic, polyethylene glycol, sucrose, and starch; disintegrants, such as starch, carboxymethyl cellulose, hydroxypropyl starch, sodium-glycol-starch, sodium hydrogen carbonate, calcium phosphate, and calcium citrate; lubricants, such as magnesium stearate, Aerosil, talc, sodium lauryl sulfate; flavors, such as citric acid, menthol, glycyrrhizic acid ammonium salt, glycine, and orange powder; preservatives, such as sodium benzoate, sodium bisulfite, methylparaben, and propylparaben; stabilizers, such as citric acid, sodium citrate, and acetates; suspensions, such as methyl cellulose, polyvinylpyrrolidone, and aluminum stearate; dispersants, such as surfactants; diluents, such as water, saline, and orange juice; base wax, such as cacao butter, polyethylene glycol, and white kerosene. The drug according to the present invention may contain other biological function-improving agents in addition to the intestinal microbe having D-psicose-responsive proliferation. Examples of such biological function-improving agents include prophylactic or therapeutic agents for metabolic disorder (e.g., anti-obesity agents, glucose tolerance improving agents) and anti-atherosclerotic agents.

When the product according to the present invention is provided in the form of food composition, the intestinal microbe having D-psicose-responsive proliferation may be provided in the state of being added to foods or may be used as a main component like supplements. Examples of foods include liquids (e.g., beverages, alcohols), semi-solids (e.g., yogurt, jelly), and solids (e.g., snacks, chocolate).

The product according to the present invention may be a composition for oral or rectal administration. Examples of the composition for oral administration include capsules, tablets, powders, and liquids. Examples of the composition for rectal administration include liquid preparations. The composition for oral administration may be preferably formulated so as to ensure good intestinal delivery. Therefore, the composition for oral administration can preferably be provided in an enteric form (e.g., enteric capsule).

Preferably, the product according to the present invention is a prophylactic or therapeutic agent for metabolic disorder. Examples of the metabolic disorder on which the intestinal microbe having D-psicose-responsive proliferation may effectively act include obesity, diabetes, arteriosclerosis, and heart failure. Therefore, the biological function-improving agent according to the present invention is useful as, for example, an anti-obesity agent, an anti-diabetic agent, an anti-atherosclerotic agent, and an anti-heart failure agent.

3. Intestinal Microbe Culturing Method

The present invention provides an intestinal microbe culturing method. The culturing method according to the present invention includes culturing intestinal microbes in a D-psicose-containing culture medium.

The intestinal microbes preferably contain the intestinal microbe having D-psicose-responsive proliferation. The intestinal microbe having D-psicose-responsive proliferation can be cultured in the form of a mixture with other intestinal microbes or in the form of a non-mixture with other intestinal microbes. For example, when the intestinal microbe having D-psicose-responsive proliferation is cultured in the form of a mixture with other intestinal microbes, such a mixture can be obtained by collection from feces of a mammal as described above.

The culturing method according to the present invention can be carried out under common culture conditions for intestinal microbes by using a culture medium similar to a common culture medium used to culture intestinal microbes except that the culture medium contains D-psicose. The concentration of D-psicose used in the culturing method according to the present invention is, for example, 0.01% to 10% (w/v), preferably 0.05% to 5% (w/v), more preferably 0.1% to 1% (w/v). The common culture medium and culture conditions for intestinal microbes are well known (e.g., (i) Fukuda S et al., J Vet Med Sci.,2002 November; 64(11): 987-92, (ii) Fukuda S et al., J Gen Appl Microbial., 2005 April; 51(2): 105-13, (iii) Sasaki. et al., Sci Rep., 2018 Jan. 11; 8(1): 435).

For example, the culture medium can contain components, such as carbon sources, nitrogen sources, organic micronutrient sources, vitamins, and inorganic ions. Examples of carbon sources include carbohydrates, such as monosaccharides (e.g., glucose), disaccharides, oligosaccharides, and polysaccharides; invert sugar, which is hydrolyzed sucrose; glycerol; compounds having one carbon atom, such as methanol, formaldehyde, formic acid salts, carbon monoxide, and carbon dioxide; oils, such as corn oil, palm oil, and soybean oil; short-chain fatty acids, such as acetates, propionates, and butyrates; organic acids, such as succinic acid and lactic acid; animal fats; animal oils; fatty acids, such as saturated fatty acids and unsaturated fatty acids; lipids; phospholipids; glycerolipids; glycerol fatty acid esters, such as monoglycerides, diglycerides, and triglycerides; polypeptides, such as microbial proteins and vegetable proteins; renewable carbon sources, such as hydrolyzed biomass carbon sources; yeast extract; horse serum; fecal extract; meat extract; vegetable extract; stomach content extract; and combinations thereof. Examples of nitrogen sources include inorganic ammonium salts, such as ammonium sulfate, ammonium chloride, and ammonium phosphate; organic nitrogen, such as hydrolyzed soybean; ammonia gas; and ammonia water. As an organic micronutrient source, for example, an appropriate amount of required substance, such as L-homoserine, or yeast extract is preferably contained. Examples of vitamins include vitamin B1, B2, B3, B6, B12, C, and K1. Examples of inorganic ions include potassium phosphate, magnesium sulfate, iron ions, and manganese ions.

With regard to the culture conditions, for example, the intestinal microbial density in the culture medium is, for example, 1×10⁶ to 1×10¹¹ cells/mL, preferably 1×10⁷ to 1×10¹⁰ cells/mL, more preferably 1×10⁸ to 1×10⁹ cells/mL. The culture temperature is, for example, 30° C. to 40° C. The culture period is, for example, 1 to 7 days. Either anaerobic culture conditions or aerobic culture conditions can be used, but anaerobic culture conditions are preferred. The oxygen concentration under anaerobic culture conditions is, for example, 0% to 5%, preferably 0% to 3%, more preferably 0% to 2%, still more preferably 0% to 1%.

The culturing method according to the present invention is useful for, for example, proliferation and maintenance of intestinal microbes having D-psicose-responsive proliferation.

The present invention also provides a culture medium that can be preferably used in the culturing method according to the present invention. The culture medium according to the present invention is a D-psicose-containing culture medium. The concentration of D-psicose is as described above. The components that may be contained in this culture medium in addition to D-psicose are the same as the components of the culture medium described above.

The present invention further provides an intestinal microbial culture that can be preferably used in the culturing method according to the present invention or obtained by the culturing method according to the present invention. The intestinal microbial culture according to the present invention contains D-psicose and the intestinal microbe. The concentration of D-psicose is the same as that described above. The intestinal microbial density is the same as the intestinal microbial density descried above in the culture medium. The components that may be contained in the intestinal microbial culture according to the present invention in addition to D-psicose and the intestinal microbe are the same as the components of the culture medium described above.

4. Screening Method for Substance having Biological Function-Improving Effects

The present invention provides a screening method for a substance having biological function-improving effects. The screening method according to the present invention includes:

(1) brining investigational substances into contact with an intestinal microbe having D-psicose-responsive proliferation;

(2) evaluating the number of the intestinal microbes having D-psicose-responsive proliferation; and

(3) selecting, as the substance having biological function-improving effects, an investigational substance that increases the number of the intestinal microbes having D-psicose-responsive proliferation.

In step (1), the investigational substances are any substances including known substances and novel substances. Examples of the investigational substances include organic low molecular weight compounds, compound libraries prepared using combinatorial chemistry techniques, nucleic acids (e.g., nucleosides, oligonucleotides, polynucleotides), carbohydrates (e.g., monosaccharides, disaccharides, oligosaccharides, polysaccharides), lipids (e.g., fatty acids containing saturated or unsaturated linear chains, branched chains, and/or rings), amino acids, proteins (e.g., oligopeptides, polypeptides, antibodies or fragments thereof), random peptide libraries prepared by solid-phase synthesis and phage display technique, or natural components from microorganisms, plants and animals, and marine organisms.

The contact between the investigational substance and the intestinal microbe having D-psicose-responsive proliferation can be carried out by any method, such as an in-vitro method or an in-vivo method.

For example, the contact between the investigational substance and the intestinal microbe having D-psicose-responsive proliferation by an in-vitro method can be achieved by culturing the intestinal microbe having D-psicose-responsive proliferation in a culture medium containing the investigational substance. The concentration of the investigational substance in the culture medium can be appropriately adjusted. For example, when screening for biological function-improving agents with high efficacy is particularly desired, the concentration of the investigational substance in the culture medium can be set to a low concentration (e.g., 1 nM to 10 μM). Alternatively, when screening for biological function-improving agents with high efficacy is not desired, the concentration of the investigational substance in the culture medium may be set to a higher concentration (e.g., 100 nM to 10 mM). The intestinal microbial density in the culture medium can be appropriately set (e.g., 1×10⁵ to 1×10³ cells/mL). The culture medium and culture conditions for the intestinal microbe may be the same as those described above in the culturing method according to the present invention.

The contact between the investigational substance and the intestinal microbe having D-psicose-responsive proliferation in an in-vivo method can be achieved by administrating the investigational substance into a mammal. The administration of the investigational substance to a mammal allows the investigational substance to come into contact with the intestinal microbe having D-psicose-responsive proliferation in the intestine of the mammal. The amount of the investigational substance to be administered can be appropriately adjusted. For example, when screening for biological function-improving agents with high efficacy is particularly desired, the amount of the investigational substance to be administered can be set to a low level (e.g., 0.05 to 10 g/kg). Alternatively, when screening for biological function-improving agents with high efficacy is not desired, the amount of the investigational substance to be administered may be set to a higher level (e.g., 10 to 100 g/kg). The mammal may be any of those described above, but preferably a primate or rodent, more preferably human or mouse. From the viewpoint of ease of clinical application, the mammal is still more preferably human.

In step (2), the number of intestinal microbes having D-psicose-responsive proliferation is evaluated. The evaluation of the number of intestinal microbes can be carried out by, for example, directly counting the number of intestinal microbes or measuring another indicator that reflects the number of intestinal microbes. Another indicator may be a marker (e.g., 16s rRNA gene, or specific substance produced by 16s rRNA gene) specific for the intestinal microbes. For example, the number of intestinal microbes can be evaluated by analyzing the 16s rRNA gene level using a quantitative method, such as quantitative PCR.

In step (3), the investigational substance that increases the number of intestinal microbes having D-psicose-responsive proliferation is selected as a substance having biological function-improving effects. The selected investigational substance can exhibit biological function-improving effects like D-psicose. Examples of the biological function-improving effects include those described for the biological function-improving agents described above.

In a specific embodiment, the screening method according to the present invention may be an in-vitro method including:

(1) brining investigational substances into contact with an intestinal microbe having D-psicose-responsive proliferation in a culture medium;

(2) evaluating the number of the intestinal microbes having D-psicose-responsive proliferation; and

(3) selecting, as a substance having biological function-improving effects, an investigational substance that increases the number of the intestinal microbes having D-psicose-responsive proliferation.

In another specific embodiment, the screening method according to the present invention may be an in-vivo method including:

(1) evaluating the number of intestinal microbes having D-psicose-responsive proliferation contained in feces of a mammal to which investigational substances have been administered; and

(2) selecting, as a substance having biological function-improving effects, an investigational substance that increases the number of intestinal microbes having D-psicose-responsive proliferation.

In another specific embodiment, the screening method according to the present invention may be an in-vivo method including:

(1′) administering investigational substances to a mammal (e.g., non-human mammal);

(2′) collecting feces of the mammal to which the investigational substances have been administered;

(3′) evaluating the number of intestinal microbes having D-psicose-responsive proliferation contained in the feces; and

(4′) selecting, as a substance having biological function-improving effects, an investigational substance that increases the number of intestinal microbes having D-psicose-responsive proliferation.

The screening method according to the present invention is useful for, for example, development of a novel drug or food having biological function-improving effects like D-psicose, and development of a drug or food having multiple effects, specifically, biological function-improving effects like D-psicose in combination with existing effects.

### EXAMPLES

Next, the present invention will be described in more detail by way of Examples. However, the present invention is not limited to the following Examples.

### Example 1

### Finding of Specific Intestinal Microbe Having D-Psicose-Responsive Proliferation

(1) Experimental Method

(a) Animal Experiment

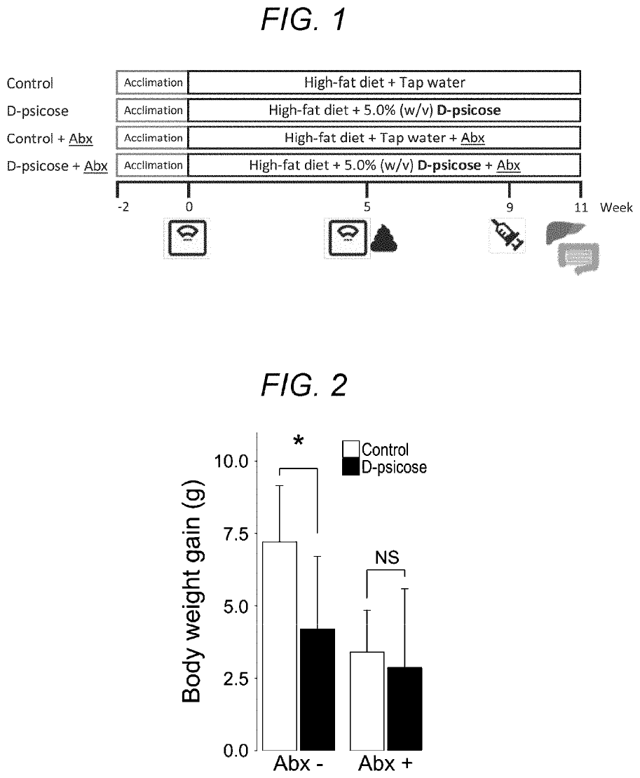

Twenty eight 6-week-old male C57BL/6J mice (CLEA Japan, Inc.) were provided and fed a normal diet CE-2 (CLEA Japan, Inc.) and tap water for 2 weeks, followed by acclimation. The mice were divided into 4 groups (7 mice per group): (i) tap water-administered group, (ii) D-psicose water-administered group, (iii) tap water and antibiotic-administered group, and (iv) D-psicose water and antibiotic-administered group. The experiment was started at 8 weeks old. For food, the mice in all groups were freely fed a high-fat diet HFD32 (CLEA Japan, Inc.). For drinking water, tap water from the laboratory was used for the tap water-administered groups, and 5.0% (w/v) D-psicose (Matsutani Chemical Industry Co., Ltd.) in tap water from the laboratory was used for the D-psicose water-administered group. To the antibiotic-administered groups, a mixture of 0.1% (w/v) ampicillin sodium, 0.1% (w/v) neomycin sulfate, and 0.05% (w/v) vancomycin hydrochloride (all of these products are available from Wako Pure Chemical Industries) with drinking water was administered. During the experimental period, the body weight of the mice, the water intake, and the food intake were measured weekly, and the feces were collected. To confirm that intestinal microbes were removed due to the effects of the antibiotics, the feces collected from the antibiotic-administered groups were suspended in PBS, applied to GAM medium (Nissui), and left to stand overnight at 37° C. under anaerobic conditions using AnaeroPack, and it was confirmed that no colonies were observed on the medium. This indicates that intestinal microbes were removed due to the effects of the antibiotics. At week 9 (17 weeks old) from the start of the experiment, the fasting blood glucose level, an indicator of diabetes, was measured. After 15-hour fasting, the side of the tail was pricked with a syringe needle (Terumo Corporation), and the blood glucose level was measured by using a glucometer (Nipro StatStrip XP3). After 11 weeks (19 weeks old) from the start of the experiment, the mice was dissected. The outline of the animal experiment is shown (FIG. 1).

(b) DNA Extraction

Fecal samples were lyophilized for 24 hours or longer and disrupted with four ϕ 3 mm zirconia beads (TOMY) in a 2 mL crushing tube using Shake master Neo (available from Bio-Medical Science Co., Ltd.) at 1,500 rpm for 10 minutes. From the fecal samples, 10 mg of fecal samples were weighed, and about 100 mg of ϕ 0.1 mm zirconia beads (TOMY), 400 μL of 1% SDS, and 400 μL of phenol/chloroform/isoamyl alcohol (25:24:1) were added. The samples were stirred using Shake master Neo at 1,500 rpm for 5 minutes and then centrifuged at 17,800×g at room temperature for 5 minutes. To the supernatant, phenol/chloroform/isoamyl alcohol (25:24:1) was added again and stirred for 1 minute using Micro mixer E-36 (TAITEC). The mixture was then centrifuged at 17,800×g at room temperature for 5 minutes. To the supernatant, 40 μL of 3M sodium acetate and 800 μL of 100% EtOH were added, and the mixture was then left to stand at −80° C. for 1 hour. After the mixture was centrifuged at 17,800×g at 4° C. for 10 minutes, EtOH was removed, and 500 μL of 70% EtOH was added. After the mixture was centrifuged again at 17,800×g at 4° C. for 10 minutes, the mixture was dried using Micro Vac (Tomy Seiko Co., Ltd), and 100 μL of TE10 was added, followed by stirring. To 80 μL of the resulting DNA solution, 1 μL of Rnase (10 mg/ml) was added, and the mixture was incubated overnight at 37° C. To the mixture, 120 μL of pure water and 200 μL of phenol/chloroform/isoamyl alcohol were added and stirred for 1 minute using Micro mixer E-36. The mixture was centrifuged at 17,800×g at room temperature for 5 minutes. To the supernatant, 20 μL of 3M sodium acetate (pH 5.2) and 400 μL of 100% EtOH were added, and the mixture was left to stand on ice for 15 minutes and then centrifuged at 17,800×g at 4° C. for 10 minutes. After the supernatant was removed, 500 μL of 70% EtOH was added. The mixture was centrifuged twice at 17,800×g at 4° C. for 10 minutes. After the supernatant was removed, 50 μL of 1×TE buffer was added and stirred for 1 minute using Micro mixer E-36 to dissolve DNA.

(c) PCR Using Universal Primer and Purification and Concentration Quantification of PCR Product

PCR was performed using 1 μL of 10 ng/μL diluted DNA solution sample as a template and using universal primers 27Fmod:5′-AGRGTTTGATYMTGGCTCAG-3′ (SEQ ID NO:2) and 338R:5′-TGCTGCCTCCCGTAGGAGT-3′(SEQ ID NO:3), which specifically amplify the V1 to V2 regions of the 16S rRNA gene of the microbe. The initial denaturation reaction was carried out at 98° C. for 1 minute, followed by 20 cycles of 3 steps of 98° C. for 10 seconds, 55° C. for 15 seconds, and 68° C. for 30 seconds. The final extension reaction was carried out at 68° C. for 3 minutes. Tks Gflex DNA Polymerase (Takara Bio Inc.) was used as a polymerase. Subsequently, the PCR product was purified by adding 81 μL of Agencourt AMPure XP (Beckman Coulter) to 45 μL of the PCR product, and the DNA concentration was measured using Picogreen (Thermo Fisher Scientific).

(d) Index PCR, and Purification, Concentration Quantification, and Sequencing of PCR Product

The molar concentration of the PCR product was calculated from the result of (c). The samples were diluted such that the concentration of the template used in index PCR was 1 nM. Index PCR was performed in order to add adapter sequences and index sequences required for sequencing in MiSeq (Illumina). Primers were 5′-AATGATACGGCGACCACCGAGATCTACAC-NNNNNNNN-TATGGTAATTGTAGRGTTTGATYMTGGCTCAG-3′ (SEQ ID NO:4) and 5′-CAAGCAGAAGACGGCATACGAGAT-NNNNNNNN-AGTCAGTCAGCCTGCTGCCTCCC GTAGGAGT-3′ (SEQ ID NO:5). The base sequences denoted by a string of Ns are index sequences and different for each sample. The index PCR conditions were as follows: the initial denaturation reaction was carried out at 98° C. for 1 minute, followed by 8 cycles of 3 steps of 98° C. for 10 seconds, 55° C. for 15 seconds, and 68° C. for 30 seconds, and the final extension reaction was carried out at 68° C. for 3 minutes. Tks Gflex DNA Polymerase was used as a polymerase. Subsequently, the PCR product was purified by adding 90 μL of Agencourt AMPure XP to 50 μL of the PCR product, and the DNA concentration was measured using Picogreen. From this result, each sample was diluted to 4 nM. Subsequently, equal volumes of the samples after the pretreatment step were mixed, and sequencing was performed in accordance with the standard protocol for MiSeq (Illumina).

(e) Data Analysis

First, base sequences sequenced by paired-end sequencing were assembled by FLASH (version 1.2.11). The base sequences with an average Q-value of 25 or less were excluded from the target of analysis. The obtained data were analyzed using Quantitative Insights into Microbial Ecology (Qiime) (version 1.9.1). The base sequences were clustered into operational taxonomic units (OTUs) at 97% or more identity. The closely related species of each OTU were estimated on the basis of the comparison with the database of Ribosomal Database Project (RDF) and the database of NCBI (16S ribosomal RNA sequences (Bacteria and Archaea)).

(f) Statistics

Statistical analysis was performed using statistical software R (version 3.4.1). First, it was confirmed that the data followed normal distribution, and if two groups were assumed to be homoscedastic, the Student's t-test was performed; otherwise, the Welch's t test was performed. If the data did not follow normal distribution, the Mann-Whitney U test (Wilcoxon rank-sum test) was performed. In any test, a p-value of 0.05 or less was considered significant.

(2) Results

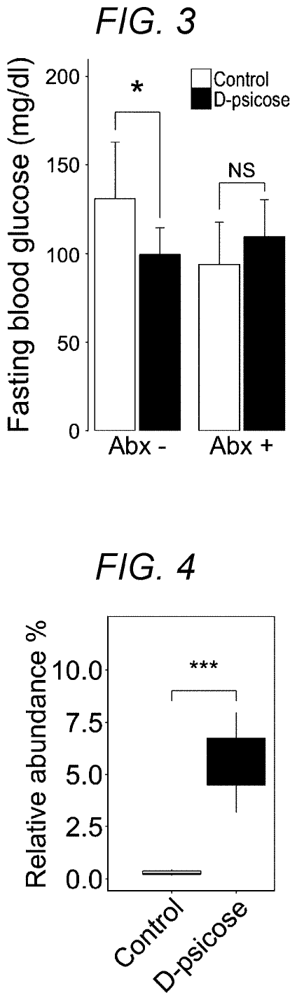

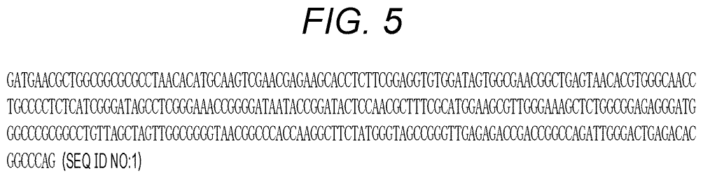

The results of weight body measurement reveal that D-psicose significantly suppresses the body weight gain due to the intake of high-fat diet. In two groups in which intestinal microbes are all removed by using a mixture of three antibiotics, the suppression of body weight gain by D-psicose is not observed (FIG. 2). Similarly, the results of fasting blood glucose level, an indicator of diabetes, reveal that the intake of D-psicose significantly reduces the blood glucose level increased by the intake of high-fat diet. There is no significant difference between the groups using antibiotics as in the results of body weight (FIG. 3). The above results indicate that the anti-obesity effect of D-psicose may be mediated via intestinal microbes. Furthermore, the analysis of intestinal microbiota in the fecal samples using next generation sequencer MiSeq indicates that the intestinal microbe found in the present invention significantly grows with the intake of D-psicose (FIG. 4).

The intestinal microbe found in the present invention has the base sequence (SEQ ID NO:1) of the 16S rRNA gene as an OTU. This base sequence was analyzed by blastn of NCBI for the database “16S ribosomal RNA sequences (Bacteria and Archaea)”, and the microbial species with the highest similarity was Atopobium parvulum (identity: 89%). Also, Olsenella umbonata has the same identity (identity: 89%). The genus Olsenella and the genus Atopobium are closely related species belonging to the same family. The genus Atopobium and the genus Olsenella are both intestinal microbes belonging to the family Atopobiaceae. In this analytical method, an identity of 97% or more is considered to be “the same species”, but the intestinal microbe found in the present invention shows 89% identity at maximum. This suggests that the intestinal microbial species obtained in this study may be an unregistered intestinal microbial species belonging to the family Atopobiaceae.

### Example 2

### Culturing of Specific Intestinal Microbe having D-Psicose-Responsive Proliferation

An intestinal microbial group containing a specific intestinal microbe having D-psicose-responsive proliferation was collected from feces of D-psicose-administered mice (Example 1) in accordance with the reference document (Fukuda S. J Vet Med Sci., 2002 November; 64(11): 987-92). The collected intestinal microbial group was cultured in a D-psicose-containing culture medium under anaerobic conditions (oxygen concentration: 0%) at 37° C. for two days. The intestinal microbial density in the culture medium at the beginning of culturing was about 1×10⁶ cells/mL. The concentration of D-psicose in the culture medium was 0.3% (w/v). Culture medium components in addition to D-psicose were the basic components for culture media described in Fukuda S. et al., J Gen Appl Microbiol. 2005 April; 51(2): 105-13, and short-chain fatty acids and vitamins described in Ohkawara S. et al., J Nutr. 2005 December; 135(12): 2878-83 (pH 7.0).

As a result, the presence of a specific intestinal microbe having D-psicose-responsive proliferation was confirmed by metagenomic analysis using the base sequence of SEQ ID NO:1.

This indicates that a specific intestinal microbe having D-psicose-responsive proliferation can be cultured in a D-psicose-containing culture medium.

### Example 3

### Culturing of Specific Intestinal Microbe having D-Psicose-Responsive Proliferation

(1) Summery

The results of Examples 1 and 2 show the suppression of body weight gain due to rare sugar intake and the characteristic growth of the Atopobium-related microbe. The anti-obesity effect of the intestinal microbe that showed a characteristic growth in the D-psicose administration test was specifically studied by producing gnotobiotic mice by administration of an Atopobium-related microbe to germ-free mice to colonize the Atopobium-related microbe.

(2) Method

(a) Microbial Broth

In this test, Atopobium parvulum (JCM10300) closely related to the intestinal microbe that grew with D-psicose administration was used. A non-administration group and a Bacteroidetes thetaiotaomicron (JCM5827)-administered group were used as control groups. Using 10 mL of GAM broth, Bacteroidetes thetaiotaomicron was cultured under anaerobic conditions for one day, and Atopobium parvulum for two days.

(b) Animal Test

Eight-week-old male C57BL/6J germ-free mice were divided into 3 groups (5 mice per group): i) germ-free group, ii) Bacteroides thetaiotaomicron-colonized group, and iii) Atopobium parvulum-colonized group. Aliquots (200 μL) of the freshly prepared microbial broth were orally administered. In all groups, the mice were freely fed a gγ-ray-sterilized high-fat diet D12492 (Research Diets, Inc.: high-fat diet, 60 kcal % fat, containing lard) as food, and sterilized tap water as drinking water. During the experimental period, the body weight of the mice, the water intake, and the food intake were measured weekly, and the fecal samples were collected. After 3, 5, 7, 14, and 21 days after microbial broth administration, the fecal samples were cultured in GAM plate culture medium, and it was confirmed that the microbe was colonized. After 9 weeks (17 weeks old) from the start of the experiment, the mice was dissected under isoflurane anesthesia.

(c) Statistics

Statistical analysis was performed using statistical software R (version 3.4.1). When a significant difference with P<0.05 was observed by two-way analysis of variance (two-way ANOVA), multiple comparison was performed by the Tukey's test.

(3) Results

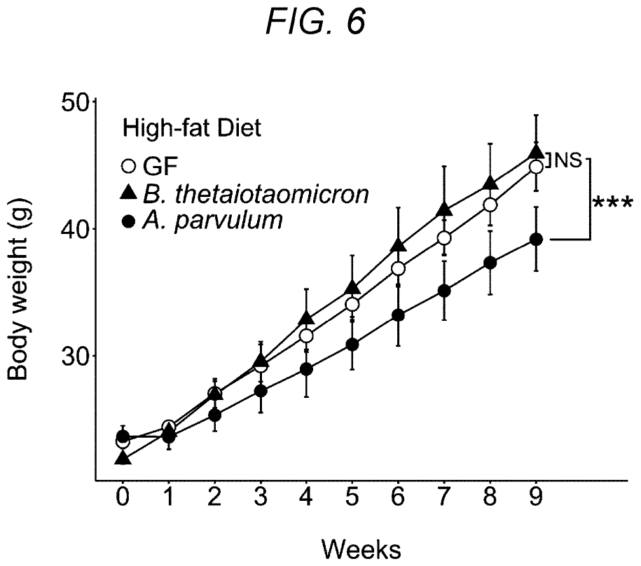

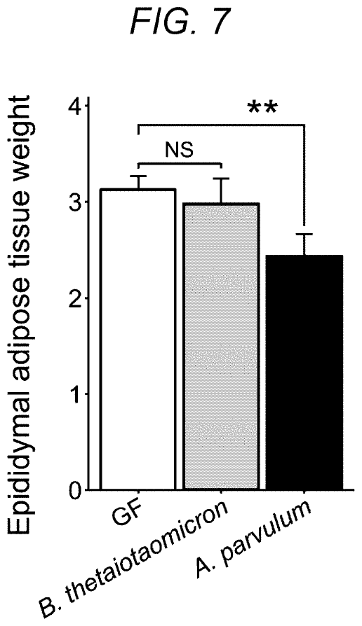

The results of body weight measurement reveal that the body weight gain due to the intake of high-fat diet is significantly suppressed in Atopobium parvulum-colonized mice compared with the germ-free group and Bacteroidetes thetaiotaomicron-colonized mice (FIG. 6). The same tendency was observed for the epididymal adipose tissue weight at the time of dissection (FIG. 7). This indicates that the Atopobium-related microbe that grows in the intestine with the intake of D-psicose has an anti-obesity effect.

The present disclosure includes an invention relating to intestinal microbial growth enhancing agent including D-psicose for enhance the proliferation of specific intestinal microbe. As described in the above examples, D-psicose has an effect to enhance the proliferation of the intestinal microbe belonging to the family Atopobiaceae. The disclosure of intestinal microbial growth enhancing agent for intestinal microbe belonging to the family Atopobiaceae may be a composition for oral or rectal administration. That is, in addition to oral administration to mammals, the product according to the present invention may be administered as enteral nutrition, adding the product as active ingredient into enteral nutrient and administered via tube inserted into the stomach or small intestine.

The form of the intestinal microbial growth enhancing agent of the present disclosure is not limited as long as it contains D-psicose as active ingredient. As an example, in addition to those consisting only of D-psicose, other components may be added as appropriate to form pharmaceuticals, food additives, supplements, and the like. In the form of pharmaceuticals, food additives, and supplements, the dosage forms include, for example, powders, tablets, sugar coatings, capsules, granules, dry syrups, liquids, syrups, drops, drinks, etc. The pharmaceuticals, food additives, and supplements of each of the above dosage forms can be produced by a method known to those skilled in the art.

The present disclosure also includes an invention relating to biological function-improving agent containing D-psicose and an intestinal microbe belonging to the family Atopobiaceae. The biological function-improving agent according to the present disclosure is a prophylactic or therapeutic agent for metabolic disorder, particularly anti-obesity agent or anti-diabetic agent. The biological function-improving agent according to the present disclosure is not limited in its form as long as it contains D-psicose and an intestinal microbe belonging to the family Atopobiaceae. As an example, in addition to those consisting only of D-psicose as active ingredient and an intestinal microbe belonging to the family Atopobiaceae, other ingredients may be added as appropriate to form pharmaceuticals, food additives, supplements, and the like. It can be produced by a method known to those skilled in the art.

1. An intestinal microbe growth enhancing agent comprising D-psicose, wherein the intestinal microbe belongs to the family Atopobiaceae.

2. The agent according to claim 1, wherein the intestinal microbe belonging to the family Atopobiaceae is Atopobium parvulum.

3. The agent according to claim 1, wherein the intestinal microbe growth enhancing agent is a composition for oral or rectal administration.

4. The agent according to claim 2, wherein the intestinal microbe growth enhancing agent is a composition for oral or rectal administration.

5. A biological function-improving agent comprising D-psicose and an intestinal microbe belonging to the family Atopobiaceae.

6. The agent according to claim 5, wherein the biological function-improving agent is a prophylactic or therapeutic agent for metabolic disorder.

7. The agent according to claim 5, wherein the biological function-improving agent is an anti-obesity agent.

8. The agent according to claim 5, wherein the biological function-improving agent is an anti-diabetic agent.

9. The agent according to claim 5, wherein the biological function-improving agent is a composition for oral or rectal administration.

10. The agent according to claim 6, wherein the biological function-improving agent is a composition for oral or rectal administration.

11. The agent according to claim 7, wherein the biological function-improving agent is a composition for oral or rectal administration.

12. The agent according to claim 8, wherein the biological function-improving agent is a composition for oral or rectal administration.
